(19) United States
(12) Patent Application Publication
(10) Pub. No.: US 2014/0148881 A1
(43) Pub. Date: May 29, 2014

(54) APPARATUS AND METHOD FOR CONTROLLING A BODY TEMPERATURE

(71) Applicant: SEIRATHERM GmbH, Herzogenaurach (DE)

(72) Inventors: Matthias ROTH, Giggenhausen (DE); Marc BAENKLER, Herzogenaurach (DE)

(73) Assignee: SEIRATHERM GmbH, Herzogenaurach (DE)

(21) Appl. No.: 14/059,226

(22) Filed: Oct. 21, 2013

Related U.S. Application Data
(63) Continuation of application No. PCT/EP2012/057223, filed on Apr. 19, 2012.

(30) Foreign Application Priority Data
Apr. 19, 2011 (EP) 11163052.1

(51) Int. Cl. A61F 7/00

(52) U.S. Cl. CPC A61F 7/0085
USPC 607/105

(57) ABSTRACT

A device and a method for controlling a temperature of a patient by an infusion of fluid. The device includes at least one supply of infusion fluid; at least one body temperature input adapted to receive the actual body temperature of the patient and at least one additional input adapted to receive at least one additional parameter representing the actual physiological state of the patient. Furthermore, the device includes at least one control unit communicating with the body temperature input, and the additional input and at least one actuator, which is in fluid communication with the supply and which controls at least the actual flow rate and/or actual temperature of the infusion fluid in accordance with at least one control signal of the control unit. The method controls a temperature of a patient by an infusion of fluid using the device.

This nonprovisional application is a continuation of International Application No. PCT/EP2012/057223, which was filed on Apr. 19, 2012, and which claims priority to European Application No. 11163052.1, which was filed in Germany on Apr. 19, 2011, and which are both herein incorporated by reference.

### BACKGROUND OF THE INVENTION

1. Field of the Invention

The present invention relates to an apparatus and a method for controlling the temperature of a patient body, wherein the infusion of a fluid is used for controlling the body temperature.

2. Description of the Background Art

The positive effect of hypothermia, i.e. the systematical lowering of the body temperature, has been investigated in many studies. The result suggests reduced tissue damage and an improvement of the patient-outcome as a consequence of lowering the body temperature during the acute phase of a patient. The basic mechanism is the general decrease of the reaction rate of (bio-)chemical reactions due to lowered temperatures (van 't Hoffs rule). A typical mode of application may include lowering the body temperature by e.g. 3 degrees C. and sustaining the lowered temperature over several hours. e.g., induced (i.e. iatrogenic) hypothermia has already become a worldwide standard by evidence based medicine criteria for patients in post-reanimation state. Thanks to the simple application, robustness and effectiveness, successful applications of induced hypothermia may also be expected in connection with the treatment of ischemic apoplexia as animal experiments already suggest. A positive effect can also be expected for acute care of trauma patients. To avoid shivering, medication suppressing the trembling may be administered.

Beside induced hypothermia, deliberate adjustment of the body temperature can be a successful approach of fighting hyperthermic states, such as fever.

Further, there may be other advantageous applications of adjusting body temperature by heating, such as regaining normothermia for a person suffering hypothermia or being subject to cooling therapeutic measures.

The technical means known from the conventional art in order to adjust the body temperature of a patient include cooling (or heating) blankets and cooling sleeves. With the devices, known from the prior art, it is difficult or not possible at all to maintain a sufficiently stable body temperature of a patient. For example, surface cooling devices such as fans, particularly cooling blankets and cooling sleeves are not effective if a large percentage of the body area is bandaged. Further, they may hamper other actions performed by medical personnel, for example if the position of the patient needs to be changed or if a body area covered by such a cooling device needs to be accessed. Moreover, surface cooling techniques are generally labour intensive and allow changes in the body temperature at moderate speed.

Endovascular cooling for instance conducted with cooling catheters is also known from the prior art. The heat is transferred from central venous blood through the wall of a central venous catheter to a heat carrier medium inside the catheter. This method is highly invasive and the surface limits its effectiveness due to limited heat transfer area of the catheter. Moreover, the period of application for one catheter is limited as the thrombosis risk will increase over time.

For a more rapid induction of hypothermia, chilled intravenous fluids (e.g., normal saline) are infused. In patients with postanoxic encephalopathy, subarachnoid hemorrhage, or traumatic brain injury, the infusion of large volumes of fluids is generally tolerated well and is considered as safe for patients with acute stroke. The infusion fluids usable for induction of hypothermia are for instance regular isotonic sodium-chloride fluid as well as almost the entire variety of fluids to be infused into emergency and intensive care patients anyway for various medical purposes, such as compensation of fluid loss, providing nutrients, administering pharmaceuticals etc.

One device for the infusion of fluids is described in WO 2009/056640. The document discloses an apparatus and method for adjusting or stabilizing the body temperature of a patient by an actively controlled infusion of fluid of a preferably known and/or controlled temperature, employing a feedback control with body temperature being a measured variable and fluid flow being an actuating variable. According to one embodiment, the adjustment and stabilization of the body temperature of a patient is achieved by an actively controlled balance of volume flows of infusion fluids provided at temperatures differing from one another. The volume flows result in a combined volume flow at a suitable temperature being continuously infused into the patient. The controller of the apparatus controls the infusion of fluid dependent on a variety of input parameters each of them related to a temperature or a flow rate. The temperatures considered are for instance the temperature of the patient, an admissible minimum and maximum temperature of the infusion(s) as well as the ambient temperature. The considered flow rates are for instance the total flow rate of fluid given to a patient or the different flow rates of the different infusions in use. The control unit uses target settings such as for instance a required body temperature and a daily infusion amount entered by a user.

Document WO2007/078463 A1, which corresponds to U.S. Pat. No. 8,343,097, and which relates to a system for infusing a cooled infusate to a target location in a patient for selective organ cooling. The temperature of the blood and infusate admixture upstream is monitored and a feedback system is utilized to control the volume, temperature, and infusion rate of the infusate. The system monitors or calculates hematocrit and adjusts infusion.

Document US 2005/0107741 A1 discloses a system for controlling the temperature of the target tissue region. The system includes a catheter and a control system that controls the amount of cooling fluid and blood provided to the tissue region. The control system adjusts the cooling procedure according to an information about the patient's physiology such as the heart rate, heart rhythm, blood pressure, blood oxygen level.

Document WO2008/124644 A1, which corresponds to U.S. Pat. No. 8,480,648 and U.S. Pat. No. 8,100,880, and which is directed to an automated therapy system having an infusion catheter; a sensor adapted to sense a patient parameter; and a controller communicating with the sensor and programmed to control flow output from the infusion catheter into a patient based on the patient parameter without removing fluid from the patient. The rate of fluid infusion and fluid temperature is adapted in accordance with a parameter.

These documents are, inter alia, limited to calculate and provide the required total amount of cooling without differentiating how the infusion parameters itself should be adjusted in order to provide the total amount of cooling in a way which is most suitable to the patient. Moreover, these documents relate to devices to assess the condition of a patient at a present stage.

### SUMMARY OF THE INVENTION

It is therefore an object of the present invention to overcome the drawbacks of the prior art and to provide an improved and/or alternative and/or additional method or device for controlling or adjusting a temperature of a patient.

In an embodiment of the present invention, a temperature control device and method for critically ill patients is provided. Another aspect of the present invention is to provide an efficient temperature control preferably allowing relatively quick changes of the body temperature. Yet another object of the present invention is to provide a temperature control, which controls or helps to control the body temperature very precise with preferably minor deviations from a desired target body temperature. Yet another object of the present invention is to provide a temperature control which may be used in a wide temperature range. Yet another object of the present invention is to provide a safe temperature control system, which preferably may be operated without ongoing observation by clinical staff. Another aspect of the present invention is to provide a temperature control which is easy to operate, particularly easy to set up. Yet another object is to provide a temperature control which may be operated and preferably be produced at low costs. Yet another object of the present invention is to adapt the temperature body in a way which is most suitable for the patient in view of his physiological condition. Yet another object is to propose a device adapted to forecast the condition and/or the cooling more accurate. The term body relates to a body of a human or an animal.

The temperature of a patient may be controlled, alone or at least inter alia, with a device for controlling or assisting to control a temperature of a patient by an infusion of fluid or infusate. The device comprises at least one supply of infusion fluid, at least one body temperature input, at least one additional input, at least one control unit, and at least one actuator. The at least one body temperature input is adapted to receive the actual body temperature Tb of the patient and the at least one additional input adapted to receive at least one additional parameter or value AP representing the actual physiological state of the patient. The at least one control unit communicates with the body temperature input and the additional input. The at least one actuator controls or manipulates or sets or actuates or adjusts at least the actual flow rate FR and/or the actual temperature of the infusion fluid to be infused into the patient in accordance with at least one control signal of the control unit.

The control unit may adjust or control via the actuator at least one infusion parameter, preferably the flow rate FR and/or the temperature of the infusion fluid, in accordance with the target body temperature Tb, target, preferably a preset or predefined target body temperature Tb, target, preset and/or a target body temperature profile.

In the event of a target body temperature below the actual body temperature Tb, the control unit may be adapted to control the actuator so as to reduce the flow rate FR and to reduce the temperature of the infusion fluid thereby keeping the actual body temperature Tb at the target body temperature when the at least one additional parameter AP, preferably the reduction rate RR, the absorbing capacity AC and/or the patient-state-index PSI, and most preferably the change of heart rate, central venous pressure and/or amount of extravascular lungwater, is/are above a first upper threshold value, and/or to increase the flow rate FR and to increase the temperature of the infusion fluid thereby keeping the actual body temperature Tb at the target body temperature when the at least one additional parameter AP, preferably the reduction rate RR, the absorbing capacity AC and/or the patient-state-index PSI, and most preferably the change of heart rate, central venous pressure and/or amount of extravascular lungwater, is/are below a first lower threshold value. The threshold value(s) may vary for each patient.

Thus, the device not only provides the required amount of cooling to the patient in order to achieve the target temperature but also adapts the infusion parameters in accordance with the physiological state of the patient. Namely, the device, e.g., verifies based on the at least one additional parameter AP whether the amount of cooling should be provided with a moderate temperature difference between the fluid and the body temperature Tb at a higher flow rate, or whether the amount of cooling should be provided at a higher temperature difference between the fluid and the body temperature Tb at a moderate flow rate. Thereby, the device advantageously provides to the patient the required amount of cooling in a more suitable manner. Advantageously, the patient suffers less. Another preferred advantage is that the device might still be operable in particular critical situations in an automated fashion.

The volume or fluid load evaluation or correction via the flow rate FR of the infusion fluid is of higher priority than the adaption of the actual temperature of the infusion fluid. The device is preferably adapted to determine the fluid load or absorbing capacity by the feedback of the at least one additional parameter AP. The admissible temperature of the fluid is calculated based on the admissible volume and is limited by threshold values (for instance, too cold infusions may cause arrhythmias and too warm infusions may lead to an increasing inter cranial pressure above the admissible threshold value or the temperature rise might be too quick).

The at least one additional parameter (AP) representing the actual physiological state of the patient include central venous pressure and/or change of heart rate. The central venous pressure particularly indicates the present fluid load of the patient. Moreover, the change of heart rate preferably indicates the development of the cardiovascular circulation as well as of the fluid load. These parameters, particularly as gradients or time derivatives, advantageously allow to forecast the cooling (more) accurately.

The one or more additional parameter(s) AP representing the actual physiological state of the patient may be for instance a core vital parameter(s) of the patient in addition to the body temperature of the patient. In other words the at least one additional parameter AP representing the actual physiological state of the patient does not comprise the actual body temperature Tb of the patient.

It may be thereby possible to control the temperature of a patient not only by taking into account the body temperature of the patient but also other parameter(s) of the patient. The body temperature advantageously may thereby be controlled more precisely.

For a better forecasting of the patients physiological condition, the at least one additional physiological parameter may also represent or indicate the dynamic reaction of the body. In other words, not only the actual physiological condition, but preferably also an indication of the development of the physiological condition may be derivable from the at least one additional physiological parameter AP. For instance, the at least one additional parameter AP may be a first or second (time) derivative of a physiological parameter. More preferably, the at least one additional parameters AP may be the change of heart rate. More precisely, considering at least one additional physiological parameter AP representing the dynamic reaction of the body advantageously enables a more precise temperature control and a better forecast of the treatment. For instance, the fluid consumption and a potential critical state of the patient may be forecasted with higher accuracy.

The control unit of the device may control the actuator based on at least a target body temperature Tb, target, the actual temperature Tfa and/or the defined temperature Tfd of the infusion fluid, the actual body temperature Tb, the actual flow rate FR and at least one additional parameter AP representing the actual physiological state of the patient.

Controlling the at least one actuator also comprises for instance manipulating, setting, actuating, adapting and/or adjusting the actuator and thereby the physical infusion parameter(s) of the infusion fluid such as the actual flow rate FR and/or actual temperature of the infusion fluid influenced by the at least one actuator. Thereby the actual body temperature Tb of the patient may be controlled, preferably in a closed loop operation. Representing the actual physiological state of the patient may, for instance, comprise indicating the physiological state of the patient, for instance, by a direct functional relationship between the physiological state of the patient and the at least one additional parameter.

The device may comprise at least one fluid temperature input communicating with the control unit. The at least one fluid temperature input may be adapted to receive the actual temperature Tfa of the infusion fluid. Alternatively or additionally, the device may comprise a defined value for the temperature Tfd of the infusion fluid. The defined value for the temperature of the fluid may be any estimated, preset or determined value derived in a conventional way. The defined value for the temperature of the infusion fluid may for instance be entered by the user.

The device may comprise at least one flow rate input communicating with the control unit. The at least one flow rate input may be adapted to receive the actual flow rate FR of the infusion fluid. The actual flow rate may be provided by any kind of conventional flow sensing device. Alternatively or additionally, the device may, for instance, derive or calculate or estimates the actual flow rate FR of the infusion fluid based on the settings of the actuator. The device may, for instance, relate a certain value for the throttle opening of the actuator to certain flow rate at a certain fluid pressure. The device may, for instance, relate a certain pump motor speed to a certain flow rate at a certain fluid pressure. The fluid pressure may be considered as constant. Alternatively, also the pressure may be considered for the determination of the actual flow rate of the infusion fluid.

The control unit may comprise a memory adapted to store data, preferably at least the actual temperature Tfa of the infusion fluid and/or the defined temperature Tfd of the infusion fluid, the actual body temperature Tb, the actual flow rate FR, the at least one additional parameter AP representing the actual physiological state of the patient, at least one parameter defined by the user, and/or pre-defined data set by the manufacturer.

The memory of the device may store all actual and/or historical data available. This may be, for instance, any actual and/or historical data of: the flow rate FR, for instance as an accumulated or extrapolated volume flow, for instance measured per 24 hours, per hour, and/or per second; the temperature Tfa of the infusion fluid; the defined temperature Tfd of the infusion fluid; the change rate of body temperature Tb, for instance the cooling rate; the body temperature Tb, for instance a profile of the body temperature exemplified as a function body temperature versus time; the values of the at least one additional parameter AP; any other parameter directly or indirectly impacting the temperature control of a patient such as humidity, room temperature; and/or any other actual or historical data.

The memory of the device may store parameter(s) entered by a user and/or manufacturer such as: (a) target or threshold value(s) of infusion parameter(s), such as: the maximum and/or minimum flow rate FR, for instance as an accumulated or extrapolated volume flow, for instance measured per 24 hours, per hour, and/or per second; the maximum and/or minimum actual temperature Tfa of the infusion fluid; defined temperature Tfd of the infusion fluid; the maximum and/or minimum fluid pressure; or any other target or threshold value of the infusion; b) target or threshold value(s) of the maximum and/or minimum body temperature Tb or body temperature profile; the maximum change rate of body temperature Tb, for instance a cooling rate; (c) target or threshold value(s) of additional parameter(s), such as: the maximum heart rate variability, the maximum intracranial pressure, the maximum change-rate of intracranial pressure, the maximum intracerebral pressure, and/or the maximum change rate of intracerebral pressure; and/or (d) any other value(s).

The memory of the device may store any other data such as patient specific data, for instance sex, age, etc., or other data related to the temperature control such as date, time, room temperature, humidity, etc.

The control unit may comprise a data processing unit adapted to read the data of the memory and/or of the inputs. For instance the at least one fluid temperature input, the at least one body temperature input, at least one additional input, and/or the at least one flow rate input. The data processing unit may be adapted to process the data to a control signal for the actuator.

The at least one additional parameter AP may be or may comprise an absorbing capacity AC representing the patient's capacity to absorb additional infusion fluid, a reduction rate RR representing the patient's capacity to reduce or eliminate the (infusion) fluid in the body, and/or a patient-state-index PSI.

At least three additional parameters AP can represent the actual physiological state of the patient. The three additional parameters may be the absorbing capacity AC, the reduction rate RR and the patient-state-index PSI. Preferably the three additional parameters may be the amount of extravascular lungwater, the central venous pressure and the change of heart rate.

The at least one additional parameter AP or the absorbing capacity AC may be or may comprise at least one or several of the parameters representing the fluid load of a patient such as amount of extravascular lungwater, intracranial pressure, change rate of intracranial pressure, intraabdominal pressure, change rate of intraabdominal pressure, compartment pressure, change rate of compartment pressure, heart rate, heart rate variability, blood pressure, arrhythmia, proBNP-level, BNP level, BNP level change rate, NTproBNP level, NTproBNP level change rate, ejection fraction of heart, ultrasonic filling status of heart and/or a capillary leak-index representing the value of a capillary leak syndrome, and the central venous pressure.

In an embodiment, if the parameter(s) representing the fluid load of a patient indicate(s) an increased or too high fluid load, the flow rate FR should be reduced and the temperature should be adapted if possible. For instance, if the value of any one of the following fluid load parameters: amount of extravascular lungwater; proBNP-level, BNP level, BNP level change rate, NTproBNP level, NTproBNP level change rate; ultrasonic filling status of heart; and/or central venous pressure, which are ranked according to their preferred significance starting with the parameter of highest significance, is increasing or is increased, the flow rate should preferably be decreased. Vice versa, if any one of the fluid load parameters is decreased or decreases the flow rate FR could preferably be increased.

Central venous pressure and compartment pressure correspond to the fluid state of the body. The heart rate variability indicates the ability of heart to respond to fluid load. Also the ejection fraction of heart indicates the state of the heart. Ultrasonic filling status of heart also indicates the state of heart and especially the preload. The parameters proBNP-level, BNP level, NTproBNP level, NTproBNP level change rate also represent the state of heart (meaning a possible heart insufficiency). BNP level change rate also indicates the state of heart and may also show a possible overstressig the heart by volume.

Extravascular lungwater, the change-rate of intrabdominal pressure as well as change-rate of compartment pressure may indicate fluid overload (hypervolemia). The change-rate of intracranial pressure shows the ongoing dynamic process of intracranial pressure. The intracranial pressure change is not linear to additional need of intracranial space. It is therefore advantageous to monitor the change-rate of intracranial pressure.

The capillary leak index is a pathologic indicator representing a phenomenon of critical ill patients meaning the shift of plasma or parts of plasma to areas outside of bloodvessels. Patients suffering a capillary leak syndrome need or resist more infusion fluid because of the explained shift. The absorbing capacity AC thus varies for patient suffering a capillary leak syndrome. A patient suffering capillary leak having a high volume need may have an adapted infusion temperature. Preferably, the flow rate FR will be increased in the event that a pathologic parameter increases. However, the flow rate FR might not necessarily be decreased if the pathologic parameter decreases.

The parameters heart rate, heart rate variability, blood pressure, arrhythmia, peripheral perfusion and splachnik perfusion preferably indicate the status of the cardiovascular circulation. Preferably, if those parameters indicate an increase or too high stress of the circulation the flow rate FR should be reduced and the temperature of the fluid should be adapted. For instance, if the blood pressure being considered as a significant parameter or the peripheral perfusion and/or splachnik perfusion being considered as a less significant parameter than blood pressure is/are decreasing or decreased, the flow rate FR may be increased and vice versa. Moreover, as another example, the flow rate FR may be decreased in the event of increasing arrhythmia, which are considered to be the less significant than blood pressure and peripheral perfusion and/or splachnik perfusion.

In an embodiment, the at least one additional parameter AP or the reduction rate RR may be or may comprise at least one or several of the parameters, which preferably indicate the productivity of the kidney: glomerular filtration rate, creatinin clearance, creatinin level, urea clearance, urea level, elimination of body waste, and/or elimination rate.

The glomerular filtration rate is an indicator for the kidney function and may be estimated by calculation of the amount of creatinin in blood. A low glomerular filtration rate indicates a bad kidney function. The creatinin level is also a parameter for kindey function and correlates to the glomerular filtration rate. A high creatinin level indicates a bad kidney function as a sign of reduced renal reduction. The creatinin clearance represents the ability of the kidney to reduce metabolites renal. Creatinin clearance is advantageously a much more precise indicator for reduced kidney capacity as a sign of tubular secretion. The creatinin level change shows the ability of body to eliminate creatinin as a sign of kidney function. Urea level and urea clearance indicate kidney dysfunction and especially the change of kidney dysfunction and also indicate the status of protein metabolism. The elimination rate indicates the actual elimination of the body waste. The elimination change rate also indicates the ability of the body to eliminate volume and/or metabolites (as a sign of kidney function). The glomerular filtration rate may be a parameter of first priority or highest relevance, whereas the creatinin clearance might be considered as being of second priority and medium relevance and the parameters creatinin level, urea level and urea clearance may be considered as being of third priority and lower relevance.

The peripheral perfusion represents the peripheral perfusion in accordance to the circulation, fluid status of body as a parameter of shock. Splachnik perfusion is a parameter of shock according to circulation.

In an embodiment, the reduction rate indicates the capability of the patient to eliminate fluid. A decreased reduction rate indicates a decreased productivity of the kidney. In most of such cases, the flow rate FR needs to be adapted, particularly when the fluid load is already at an increased level. For example, a decreased glomerular filtration rate, a decreased creatinin urea clearance, an increased creatinin level and/or an increased urea level may indicate that the flow rate FR should be decreased. Whether a reduction of the flow rate is possible would depend on the present values of above circulation parameters such as the heart rate. The temperature could be adapted as long as the circulation parameters are below upper threshold values. Vice versa, an increased reduction rate leads to an increased productivity of the kidney and a higher elimination rate. Consequently, the flow rate FR may be increased, if desired. Besides the above parameters of the reduction rate, any other suitable parameter indicating the elimination rate of fluid may be used.

In an embodiment, the reduction rate and its parameter(s) are the most important parameter(s) followed by the parameter(s) indicating the circulation, which are of higher priority than the parameter(s) indicating the fluid load followed by pathologic parameter(s).

In an embodiment, the at least one additional parameter AP or the patient-state-index PSI may be or may comprise at least one or several of the patient-state-index parameters PSIP, the patient-state-index parameters PSIP comprising the parameters: peripheral perfusion, splachnik perfusion, glomerular filtration rate, creatinin clearance, creatinin level, creatinin level change rate, urea clearance, urea level, urea level change rate, intracranial pressure, change-rate of intracranial pressure, change-rate of intraabdominal pressure, intraabdominal pressure, change-rate of compartment pressure, compartment pressure, heart rate, heart rate variability, arrhythmia, BNP level, BNP level change rate, NTproBNP-level, NTproBNP level change rate, ejection fraction of heart, ultrasonic filling status of heart, elimination, of body waste and/or elimination change rate.

In an embodiment, the data processing unit may be adapted to calculate the patient-state-index PSI. The calculation comprises the assessment, prioritization and/or weighting of at least two or several or all patient-state-index parameters PSIP. The calculation could be any mathematical operation including estimations suitable to derive to an assessment and/or weighting.

In an embodiment, the calculation of the patient-state-index may be, preferably continuously, done by an assessment of the core physiological parameters of the patient, for instance the heart rate and alteration of the heart rate, arterial oxygen saturation, central venous pressure, intracranial pressure, temperature as measured arterial, rectal, dermal, and/or shivering effects.

In an embodiment, the data processing unit may be adapted to calculate at least one additional parameter AP representing the actual physiological state of the patient, the calculation comprises the assessment, prioritization and/or weighting of at least two of the parameters: absorbing capacity AC, reduction rate RR and/or patient-state-index PSI or parameters thereof. Thus the calculation comprises the assessment, prioritization and/or weighting of at least two of the parameters: amount of extravascular lungwater, central venous pressure, intracranial pressure, intraabdominal pressure, compartment pressure, heart rate, heart rate variability, blood pressure, arrhythmia, proBNP-level, ejection fraction of heart, ultrasonic filling status of heart and/or a capillary leak-index representing the value of a capillary leak syndrome; glomerular filtration rate, creatinin clearance, creatinin level, urea clearance, urea level, elimination of body waste, and/or elimination rate; peripheral perfusion, splachnik perfusion, glomerular filtration rate, creatinin clearance, creatinin level, creatinin level change rate, urea clearance, urea level, urea level change rate, intracranial pressure, change-rate of intracranial pressure, change-rate of intraabdominal pressure, intraabdominal pressure, change-rate of compartment pressure, compartment pressure, heart rate, heart rate variability, arrhythmia, BNP level, BNP level change rate, NTproBNP-level, NTproBNP level change rate, ejection fraction of heart, ultrasonic filling status of heart, elimination of body waste and/or elimination change rate; and/or any other parameter representing the actual physiological state of the patient.

For instance, the device may use one additional parameter AP which may be based one or several aforementioned parameters or the device may use several additional parameters AP of aforementioned parameters, either alone or in combination, respectively.

In an embodiment, the data processing unit may be adapted to calculate at least one additional parameter AP representing the actual physiological state of the patient, the calculation comprises the assessment, prioritization and/or weighting of at least two of the parameters: change of heart rate, central venous pressure and/or amount of extravascular lungwater.

The data processing unit may be adapted to prioritize three additional parameters AP representing the actual physiological state of the patient. For instance, the additional parameters AP amount of extravascular lungwater, central venous pressure and change of heart rate may be prioritized, and the infusion may be interrupted when at least one of the parameters is above a threshold value. Additional parameters AP of second priority may be part of the patient-state-index PSI, and the infusion may be interrupted, when the patient-state-index is above a threshold value.

The user may select and/or prioritize the parameters being an at least one additional parameter AP representing the actual physiological state of the patient and/or being part of the patient-state-index PSI. The user may, for instance, decide which additional parameter AP is a first priority additional parameter AP and which additional parameter AP is of second priority and therefore part of the patient-state-index which thus may be an index representing the state of the second priority parameters. The patient-state-index itself may be considered as having the first priority. The device may interrupt the infusion, if a additional parameter of first priority is above a threshold value.

Alternatively or additionally, the control unit may suggest a selection and/or prioritization, for instance based on an analysis of the actual available patient data. The device may preferably analyze, which additional parameters AP are received by the device from any kind of sensor or medical device. The device may have a default prioritization by the manufacturer, for instance to prioritize extravascular lungwater, central venous pressure and change of heart rate.

For instance, the target body temperature profile may be exemplified as a function of the target body temperature versus time. The actual body temperature Tb of the patient may be controlled in a closed loop operation with a preset target body temperature which varies with time. Preferably, the target body temperature profile may be a user defined profile, preferably based on at least a preset target body temperature Tb, target, preset, a change rate of the body temperature Tb, and/or a target flow rate FR. Preferably, the device calculates and/or suggests a target body temperature profile, preferably based on least a preset target body temperature Tb, target, preset, a change rate of the body temperature Tb, and/or a target flow rate FR. Preferably the target flow rate FR may be a target volume to be infused in 24 hours. Preferably, the target body temperature Tb, target, and/or the target body temperature profile may be adapted to the patient. Preferably, the infusion may be initiated with a maximum flow rate FR and/or a temperature of the fluid which is a minimum temperature in the event of cooling of a patient and a maximum temperature in the event of warming a patient up.

Alternatively or additionally at least one target temperature profile may be preset by the manufacturer. Preferably, the pre-defined data may be stored in the at least one memory and may be accessible for the data processing unit.

In an embodiment, the control unit may control via the actuator the at least one infusion parameter within at least one upper and/or lower threshold value of the at least one infusion parameter.

The threshold value(s) of the at least one infusion parameter may be: the maximum and/or minimum flow rate FR, for instance as an accumulated or extrapolated volume flow, for instance measured per 24 hours, per hour, and/or per second; the maximum and/or minimum actual temperature Tfa of the fluid to be infused; the maximum and/or minimum fluid pressure; or any other target or threshold value of the infusion.

In an embodiment, the control unit may control via the actuator the actual body temperature Tb and/or the change rate of the actual body temperature Tb within at least one upper and/or lower threshold value.

In an embodiment, the control unit may control via the actuator the at least one infusion parameter in accordance with at least one upper and/or lower threshold value of the at least one additional parameter AP, preferably, in accordance with at least one upper and/or lower threshold value of the absorbing capacity AC, reduction rate RR and/or patient-state-index PSI or parameters thereof, most preferably in accordance with at least one upper and/or lower threshold value of the change of heart rate, central venous pressure and/or amount of extravascular lungwater.

In an embodiment, the actual temperature Tfa of the fluid to be infused and/or flow rate FR of the fluid is adapted in accordance to the dynamic reaction of the body, preferably measured by change of physiological parameters, most preferably by the change of heart rate.

In an embodiment, the control unit may, automatically, adapt the target body temperature Tb and/or the target temperature profile when the actual target body temperature Tb and/or the target temperature profile cannot be achieved by a setting of the at least one infusion parameter, which is within the upper and/or lower threshold value(s) for the at least one infusion parameter, and which ensures that the at least one additional parameter and/or the body temperature change rate are within the respective upper and/or lower threshold values.

In an embodiment, the control unit may adapt the target body temperature Tb and/or the target temperature profile when the change rate of the actual body temperature Tb is not within its upper and/or lower threshold value.

In an embodiment, the at least one target value and/or at least one threshold value for the at least one additional parameter AP, for the at least one infusion parameter, for the actual body temperature Tb, and/or for the change rate of the actual body temperature Tb may be administered by the user and/or may be a pre-defined data set by the manufacturer.

The target value and/or threshold value may be based on the individual assessment of the core vital parameters. Moreover, the device may use and/or suggest the parameters for controlling of a body temperature based on settings of the device manufacturer.

The device may be operated in a manual mode wherein only the target infusion parameters are set by the user and the device infuses the infusion fluid without controlling the infusion parameters in accordance to the actual body temperature.

The system may allow the user, for instance the physician, to realize specific temperature profiles. At each point of time during treatment, the device may be able to assess the state of the patient based on the measured physiological parameters, may match this with the desired temperature profile—as set by the user—and adjust the inflow and temperature of the cold infusion. As a consequence, the device may automatically cool-down, hold and rewarm the patient according to the physicians requirements and patients vital parameters.

Further, the device may regulate the inflowing volume and temperature of the infusion to the patient based on at least one vital physiological parameter, a specific patient-state-index, the target values as set by the physician and a preferred temperature profile.

In an embodiment, in the event of a target body temperature below the actual body temperature Tb, the control unit may be adapted to control the actuator so as to reduce the flow rate FR and to reduce the temperature of the infusion fluid thereby keeping the actual body temperature Tb at a reduced target body temperature when the at least one additional parameter AP, preferably the reduction rate RR, the absorbing capacity AC and/or a patient-state-index PSI, and most preferably the change of heart rate, central venous pressure and/or amount of extravascular lungwater, is/are above a second upper threshold value. The second upper threshold values may be higher than first threshold values. It may be preferred to have at least a reduced cooling effect compared to a no cooling effect. A reduced target body temperature may be a temperature somewhere between the original target body temperature and the present body temperature.

In an embodiment, in the event of a target body temperature below the actual body temperature Tb, the control unit may be adapted to stop the supply of infusion to the patient in the event of at least one additional parameter AP, preferably the reduction rate RR, the absorbing capacity AC and/or a patient-state-index PSI, and most preferably the change of heart rate, central venous pressure and/or amount of extravascular lungwater, below a third upper threshold value. The third upper threshold value may be higher than second threshold value.

In an embodiment, the device may be adapted to trigger an alarm and/or to display an information and/or different status information when the at least one lower and/or upper threshold value is achieved. The device may, for instance display the status of the device and/or patient. Preferably the device may display different content, for instance in different colors depending on present values of the parameters. The device may comprise at least one display, for instance a human-machine-interface such as a touch panel. Besides different visual status information the device may also, additionally or alternatively, provide audible alarms. The alarm could be triggered by any kind of threshold value. Preferably the alarm may be triggered based on a threshold value for the at least one additional parameter AP, for the at least one infusion parameter, for the actual body temperature Tb, and/or for the change rate of the actual body temperature Tb.

In an embodiment, a first lower threshold value may be, for instance, a central venous pressure of 12 cm H2O, a second lower threshold value may be, for instance, a central venous pressure of 20 cm H2O, and a the third lower threshold value may be, for instance, a central venous pressure of 35 cm H2O. These first, second and third threshold values may be defined differently for each patient. Preferably, the third upper threshold value may be, for instance, a change of heart rate of 20 beats per minute with a heart beat in the range of 60 to 180 beats per minute. The values may be defined differently for each patient.

In an embodiment, the device may be adapted to trigger an alarm and/or to display an information and/or different status information when the amount of infusion left in the supply may be below a minimum value, and/or the remaining period of time until the infusion needs to be refilled may be below a minimum value. Preferably, the minimum period of time and/or minimum infusion amount may be defined by the user or the manufacturer. Preferably, the remaining period of time may be calculated or estimated by the device based on the remaining infusion amount.

The device may be adapted to allow scalable access, for instance with a patient mode which gives reading access to very few parameters such as the infusion parameters and temperature values, and with an expert mode which access to all parameters of the device. The parameters forming the patient-state-index may only be visible in the expert mode.

In an embodiment, the fluid temperature input, the body temperature input, the additional input, and/or the flow rate input may comprise at least one sensor element, and/or at least one connector for connecting at least one sensor element and/or at least one medical device. The at least one sensor element and/or at least one connector may be any kind of sensor element and/or any kind of connector adapted to receive, measure, and/or process at least the actual temperature Tfa of the infusion fluid, the actual body temperature of the patient Tb, the at least one additional parameter AP representing the actual physiological state of the patient, the actual flow rate FR and/or any other parameter, preferably any one or more of above or below parameter(s). The sensor element may, for instance, be any kind of sensing device such as a temperature or flow rate sensing device. The at least one sensor element may be connected or connectable to the device via conventional electronic cabling or may be configured as a sensor which is part of the device as an inbuilt sensor. The at least one connector of the device may be in electronic communication with a medical device indicating the actual temperature Tfa of the infusion fluid, the actual body temperature of the patient Tb, the at least one additional parameter AP representing the actual physiological state of the patient, the actual flow rate FR and/or any other parameter, preferably any one or more of above or below parameter(s). The medical device may be an intensive care monitoring device.

Therefore, the device may either directly measure core vital parameters, for instance temperature of the patient, or process data, as determined by other intensive care monitors, for instance central venous pressure, in order to assess the general state of the patient represented by the patient-state-index. The device may have an user-interface in order to set the target and/or threshold values based on the requirements, for instance target temperature, preferred temperature profile, tolerable volume, etc.

In an embodiment, the at least one actuator may be in fluid communication with the supply. Preferably, the supply may be connectable and/or may comprise at least one reservoir. Preferably, the supply may be an inlet connectable to the reservoir, preferably by an inlet tube. The reservoir may preferably be an infusion bag. The reservoir may alternatively be a separate housing, for instance a thermo box. The thermo box may comprise a container for the infusion fluid. Also, an infusion bag may be used as a container in the thermo box. Alternatively, the reservoir may be part of the supply when the reservoir is a part of the device. The supply may be in fluid communication with one or more reservoirs.

In an embodiment, the reservoir may be connectable and/or comprise at least one temperature regulating device for cooling and/or heating of the infusion of the reservoir. The temperature of at least a part of the infusion, preferably the whole infusion may be regulated by the temperature regulating device. The temperature regulating device may be any kind temperature regulating device adapted for cooling and/or heating of an infusion. The temperature regulating device may be embodied as a cooling cuff for an infusion bag. The temperature regulating device may be integrated into a thermo box. The temperature regulating device may comprise one or more thermoplates, heat exchangers, microwave ovens, cooling units, thermal packs, etc. The temperature regulating device may have a separate control unit regulating the temperature of the reservoir or may be controlled by the control unit of the device. The device may be connectable and/or may comprise at least two reservoirs of infusion fluids. Preferably, the infusion fluids of the at least two reservoirs have different temperatures. Preferably, the temperature of the infusion to be infused may at least partially be influenced by mixing the at least two infusion fluids having different temperatures.

In an embodiment, the reservoir may be connectable and/or comprise at least one supply temperature sensor. The supply temperature sensor provides preferably a temperature signal to the controller of the temperature regulating device and/or the control unit of the device. Preferably, the temperature regulating device and/or the supply temperature sensor may be in communication with the control unit of the device. Preferably, the supply temperature sensor may serve as sensor element for determining actual temperature Tfa of the infusion fluid. Preferably, the defined temperature Tfd of the infusion fluid may be entered by a user or obtained by an external device, preferably obtained by the temperature regulating device.

In one configuration of the device, no temperature regulating device may be used and the temperature of the infusion fluid may be adapted only prior to infusion by the temperature actuator of the actuator of the device.

In another configuration, only the temperature of the infusion fluid in the reservoir may be controlled by the controller of the temperature regulating device or the control unit of the device via the temperature regulating device to the defined fluid temperature Tfd and only the flow rate FR may be regulated by the actuator of the device.

In yet another configuration, the temperature of the fluid in the reservoir may be controlled by the controller of the temperature regulating device or the control unit of the device via the temperature regulating device to the defined fluid temperature Tfd or any other suitable fluid temperature and the actuator of the device additionally may control the fluid temperature of the infused fluid. The actuator of the device may, moreover, additionally also control the flow rate of the infused fluid. The latter configuration may improve the response time of the body temperature control system.

The device may thus receive two fluid temperature signals, one of the fluid to be infused and one of the stored fluid in the reservoir, and/or may actuate two temperature actuator or temperature regulating device. Such a configuration may have a better control performance than a configuration operating with only one fluid temperature signal and/or only one temperature actuator or temperature regulating device, however, the costs of such a device would also be higher.

In an embodiment, the device may comprise and/or may be connectable to at least one patient connector which may connect the actuator of the device with the patient. The device is thus in fluid communication with the patient. The connector may be an infusion needle connected to a tube.

In an embodiment, the actuator may comprise at least one temperature actuator, preferably at least one heating element, at least one cooling element, and/or at least one fluid mixing element or the like. The temperature actuator may be any kind of actuator adapted to change the temperature of the fluid to be infused. The temperature actuator may be a heat exchanger, microwave and/or a fluid mixing element in fluid communication with two fluids of different temperatures as described in WO 2009/056640.

Moreover, the actuator may comprise at least one flow rate actuator, preferably at least one valve and/or at least one pump. The flow rate actuator may be any kind of actuator adapted to change the flow rate of the fluid to be infused.

Moreover the object of the present invention is attained by a method for controlling or assisting to control a temperature of a patient by an infusion of fluid, preferably with aforementioned device for controlling a temperature of a patient by an infusion of fluid, comprising at least the steps of: providing the actual body temperature Tb of the patient; providing at least one additional parameter AP representing the actual physiological state of the patient; and controlling at least one actuator which is preferably in fluid communication with a supply of fluid and which controls the actual flow rate FR and/or actual temperature of the infusion fluid to be infused in accordance with at least one control signal of a control unit.

A device is also provided for controlling or assisting to control a temperature of a patient by an infusion of fluid, comprising: at least one supply of infusion fluid; at least one body temperature input adapted to receive the actual body temperature of the patient; at least one additional input adapted to receive at least one additional parameter representing the actual physiological state of the patient; at least one control unit communicating with the body temperature input and the additional input; and at least one actuator which controls at least the actual flow rate and/or actual temperature of the infusion fluid in accordance with at least one control signal of the control unit.

In an embodiment, the control unit controls the actuator based on at least a target body temperature, the actual temperature and/or the defined temperature of the infusion fluid, the actual body temperature, the actual flow rate and at least one additional parameter representing the actual physiological state of the patient.

In an embodiment the device comprises: at least one fluid temperature input communicating with the control unit and adapted to receive the actual temperature of the infusion fluid, and/or a defined value for the temperature of the infusion fluid.

In an embodiment the device has at least one flow rate input communicating with the control unit and adapted to receive the actual flow rate of the infusion fluid, and/or wherein the device derives the actual flow rate of the infusion fluid based on the settings of actuator.

In an embodiment, the control unit includes: a memory adapted to store data, preferably at least the actual temperature and/or the defined temperature of the infusion fluid, the actual body temperature, the actual flow rate, the at least one additional parameter representing the actual physiological state of the patient, at least one parameter defined by the user, and/or pre-defined data set by the manufacturer, and/or a data processing unit adapted to read the data of the memory and/or of the inputs and process the data to a control signal for the actuator.

In an embodiment, the at least one additional parameter comprises: an absorbing capacity representing the patient's capacity to absorb additional infusion fluid, a reduction rate representing the patient's capacity to reduce the infusion fluid in the body, and/or a patient-state-index.

In an embodiment, at least three additional parameters represent the actual physiological state of the patient, and the three additional parameters are: absorbing capacity, reduction rate and the patient-state-index.

In an embodiment, at least three additional parameters represent the actual physiological state of the patient, and the three additional parameters are: amount of extravascular lungwater, central venous pressure and change of heart rate.

In an embodiment, the at least one additional parameter or the absorbing capacity comprises at least one or several of the parameters: amount of extravascular lungwater, central venous pressure, intracranial pressure, intraabdominal pressure, compartment pressure, heart rate, heart rate variability, blood pressure, arrhythmia, proBNP-level, ejection fraction of heart, ultrasonic filling status of heart and/or a capillary leak-index representing the value of a capillary leak syndrome.

In an embodiment, the at least one additional parameter or the reduction rate comprises at least one or several of the parameters: glomerular filtration rate, creatinin clearance, creatinin level, urea clearance, urea level, elimination of physiological waste, and/or elimination rate.

In an embodiment, the at least one additional parameter or the patient-state-index comprises at least one or several of the patient-state-index parameter(s), the patient-state-index parameter(s) comprising the parameters: peripheral perfusion, splachnik perfusion, glomerular filtration rate, creatinin clearance, creatinin level, creatinin level change rate, urea clearance, urea level, urea level change rate, intracranial pressure, change-rate of intracranial pressure, change-rate of intraabdominal pressure, intraabdominal pressure, change-rate of compartment pressure, compartment pressure, heart rate, heart rate variability, arrhythmia, BNP level, BNP level change rate, NTproBNP-level, NTproBNP level change rate, ejection fraction of heart, ultrasonic filling status of heart, elimination of physiological waste, and/or elimination change rate.

In an embodiment, the data processing unit is adapted to calculate the patient-state-index, the calculation comprises the assessment, prioritization and/or weighting of at least two or several or all patient-state-index parameters.

In an embodiment, the data processing unit is adapted to calculate at least one additional parameter representing the actual physiological state of the patient, the calculation comprises the assessment, prioritization and/or weighting of at least two of the parameters: absorbing capacity, reduction rate and/or patient-state-index or parameters thereof.

In an embodiment, the data processing unit is adapted to calculate at least one additional parameter representing the actual physiological state of the patient, the calculation comprises the assessment, prioritization and/or weighting of at least two of the parameters: change of heart rate, central venous pressure and/or amount of extravascular lungwater.

In an embodiment, the data processing unit is adapted to prioritize three additional parameters representing the actual physiological state of the patient, wherein the additional parameters amount of extravascular lungwater, central venous pressure and change of heart rate are prioritized, and wherein the infusion is interrupted when at least one of the additional parameter is above a threshold value.

In an embodiment, additional parameters of second priority are part of the patient-state-index, and the infusion is interrupted when patient-state-index is above a threshold value.

In an embodiment, the user selects and/or prioritize the parameters being an at least one additional parameter representing the actual physiological state of the patient and/or being part of the patient-state-index.

In an embodiment, the control unit controls via the actuator at least one infusion parameter, preferably the flow rate and/or the temperature of the infusion fluid, in accordance with the target body temperature, preferably a preset target body temperature and/or a target body temperature profile.

In an embodiment, the target body temperature profile is a user defined profile, based on at least a preset target body temperature, a change rate of body temperature, and/or target flow rate.

In an embodiment, the control unit controls via the actuator the at least one infusion parameter within at least one upper and/or lower threshold value of the at least one infusion parameter, and/or controls via the actuator the actual body temperature and/or the change rate of the actual body temperature within at least one upper and/or lower threshold value.

In an embodiment, the control unit controls via the actuator the at least one infusion parameter in accordance with at least one upper and/or lower threshold value of the at least one additional parameter, preferably of the absorbing capacity, reduction rate and/or patient-state-index or parameters thereof, most preferably at least one upper and/or lower threshold value of the change of heart rate, central venous pressure and/or amount of extravascular lungwater.

In an embodiment, the control unit adapts the target body temperature and/or the target temperature profile when the actual target body temperature and/or the target temperature profile cannot be achieved by a setting of the at least one infusion parameter, which is within the upper and/or lower threshold value(s) for the at least one infusion parameter, and which ensures that the at least one additional parameter and/or the temperature change rate are within the respective upper and/or lower threshold value(s), and/or when the change rate of the actual body temperature is not within its upper and/or lower threshold value.

In an embodiment, at least one target value and/or at least one threshold value for the at least one additional parameter, for the at least one infusion parameter, for the actual body temperature, and/or for the change rate of the actual body temperature is/are administered by the user and/or is/are pre-defined data set by the manufacturer.

In an embodiment, in the event of a target body temperature below the actual body temperature, the control unit is adapted to control the actuator so as to reduce the flow rate and to reduce the temperature of the infusion fluid thereby keeping the actual body temperature at the target body temperature when the at least one additional parameter, preferably the reduction rate, the absorbing capacity and/or the patient-state-index, and most preferably the change of heart rate, central venous pressure and/or amount of extravascular lungwater, is/are above a first upper threshold value, and/or to increase the flow rate and to increase the temperature of the infusion fluid thereby keeping the actual body temperature at the target body temperature when the at least one additional parameter, preferably the reduction rate, the absorbing capacity and/or the patient-state-index, and most preferably the change of heart rate, central venous pressure and/or amount of extravascular lungwater, is/are below a first lower threshold value.

In an embodiment, in the event of a target body temperature below the actual body temperature, the control unit is adapted to control the actuator so as to reduce the flow rate and to reduce the temperature of the infusion fluid thereby keeping the actual body temperature at a reduced target body temperature when the at least one additional parameter, preferably the reduction rate, the absorbing capacity and/or a patient-state-index, and the change of heart rate, central venous pressure and/or amount of extravascular lungwater, is/are above a second upper threshold value.

In an embodiment, in the event of a target body temperature below the actual body temperature, the control unit is adapted to stop the supply of infusion to the patient in the event of at least one additional parameter, preferably the reduction rate, the absorbing capacity and/or a patient-state-index, and the change of heart rate, central venous pressure and/or amount of extravascular lungwater, below a third upper threshold value.

In an embodiment, the device is adapted to trigger an alarm and/or to display an information and/or different status information when the at least one lower and/or upper threshold value is achieved.

In an embodiment, a first upper threshold value is a central venous pressure of 12 cm H2O, a second upper threshold value is a central venous pressure of 20 cm H2O, and a the third upper threshold value is a central venous pressure of 35 cm H2O.

In an embodiment, the third upper threshold value is a change of heart rate of 20 beats per minute with a heart beat in the range of 60 to 180 beats per minute.

In an embodiment, the device is adapted to trigger an alarm and/or to display an information and/or different status information when the amount of infusion left in the supply is below a minimum value, and/or the remaining period of time until the infusion needs to be refilled is below a minimum value.

In an embodiment, the fluid temperature input, the body temperature input, additional input, and/or the flow rate input comprise(s): at least one sensor element, and/or at least one connector for connecting at least one sensor element and/or at least one medical device, wherein the at least one sensor element and/or at least one connector is/are adapted to receive, measure, and/or process the actual temperature of the infusion fluid, the actual body temperature of the patient, the at least one additional parameter representing the actual physiological state of the patient, the actual flow rate and/or any other parameter.

In an embodiment, the at least one actuator is in fluid communication with the supply, and/or the supply is connectable and/or comprises at least one reservoir, the reservoir baing an infusion bag.

In an embodiment, the reservoir is connectable and/or comprises at least one temperature regulating device, and/or least one supply temperature sensor.

In an embodiment, the temperature regulating device and/or the supply temperature sensor is/are in communication with the control unit.

In an embodiment, the defined temperature of the infusion fluid is entered by a user or obtained by an external device, obtained by the temperature regulating device and/or the supply temperature sensor.

In an embodiment, the device comprises and/or is connectable to at least one patient connector which connects the actuator with the patient.

In an embodiment, the actuator comprises: at least one temperature actuator, at least one heating element, at least one cooling element, and/or at least one fluid mixing element, and/or at least one flow rate actuator, at least one valve and/or at least one pump.

In an embodiment, a method is provided for controlling or assisting to control a temperature of a patient by infusion of fluid, comprising: providing the actual body temperature of the patient; providing at least one additional parameter representing the actual physiological state of the patient; controlling at least one actuator which controls the actual flow rate; and/or actual temperature of the infusion fluid in accordance with at least one control signal of a control unit.

In an embodiment, the controlling further comprises: controlling the actuator based on at least a target body temperature, the actual temperature and/or the defined temperature of the infusion fluid, the actual body temperature, the actual flow rate and at least one additional parameter representing the actual physiological state of the patient.

In an embodiment, the temperature of the infusion fluid is provided, wherein the device comprises at least one fluid temperature input communicating with a control unit and receiving the actual temperature of the infusion fluid, and/or wherein the device preferably provides a defined value for the temperature of the infusion fluid.

In an embodiment, the actual flow rate of the infusion fluid is provided, preferably received from at least one flow rate input, and/or deriving the actual flow rate of the infusion fluid based on the settings of the actuator.

In an embodiment, the method further comprises the steps of: storing data on a memory, preferably at least the actual temperature and/or the defined temperature of the infusion fluid, the actual body temperature, the actual flow rate, the least one additional parameter representing the actual physiological state of the patient, at least one target parameter defined by the user, and/or pre-defined data set by the manufacturer, and/or the step of processing the data to a control signal for the actuator with a processing unit which reads the data of the memory and/or the provided data.

In an embodiment, the at least one additional parameter comprises an absorbing capacity (AC) representing the patient's capacity to absorb additional infusion fluid, a reduction rate (RR) representing the patient's capacity to reduce the infusion fluid in the body, and/or a patient-state-index (PSI).

In an embodiment, at least three additional parameter represent the actual physiological state of the patient and wherein three additional parameters are: absorbing capacity, reduction rate and the patient-state-index.

In an embodiment, at least three additional parameter represent the actual physiological state of the patient and wherein three additional parameters are: amount of extravascular lungwater, central venous pressure and change of heart rate.

In an embodiment, the at least one additional parameter or the absorbing capacity comprises at least one or several of the parameters: amount of extravascular lungwater, central venous pressure, intracranial pressure, intraabdominal pressure, compartment pressure, heart rate, heart rate variability, blood pressure, arrhythmia, proBNP-level, ejection fraction of heart, ultrasonic filling status of heart and/or a capillary leak-index representing the value of a capillary leak syndrome.

In an embodiment, the at least one additional parameter or the reduction rate comprises at least one or several of the parameters: glomerular filtration rate, creatinin clearance, creatinin level, urea clearance, urea level, elimination, and/or elimination rate.

In an embodiment, the at least one additional parameter or the patient-state-index comprises at least one or several of the patient-state-index parameter(s), the patient-state-index parameter(s) comprising the parameters: peripheral perfusion, splachnik perfusion, glomerular filtration rate, creatinin clearance, creatinin level, creatinin level change rate, urea clearance, urea level, urea level change rate, intracranial pressure, change-rate of intracranial pressure, change-rate of intraabdominal pressure, intraabdominal pressure, change-rate of compartment pressure, compartment pressure, heart rate, heart rate variability, arrhythmia, BNP level, BNP level change rate, NTproBNP-level, NTproBNP level change rate, ejection fraction of heart, ultrasonic filling status of heart, elimination, elimination change rate.

In an embodiment, the data processing unit calculates the patient-state-index, the calculation comprises the assessment, prioritization, and/or weighting of at least one or several or all patient-state-index parameters.

In an embodiment, the data processing unit calculates at least one additional parameter representing the actual physiological state of the patient, the calculation comprises the assessment, prioritization, and/or weighting of at least two of the parameters: absorbing capacity, reduction rate and/or patient-state-index or parameters thereof.

In an embodiment, the data processing unit calculates at least one additional parameter representing the actual physiological state of the patient, the calculation comprises the assessment, prioritization, and/or weighting of at least two of the parameters: change of heart rate, central venous pressure and/or amount of extravascular lungwater.

In an embodiment, the data processing unit is adapted to prioritize three additional parameters representing the actual physiological state of the patient, wherein the additional parameters amount of extravascular lungwater, central venous pressure and change of heart rate are prioritized, and wherein the infusion is interrupted when at least one of the additional parameter is above a threshold value.

In an embodiment, the additional parameters of second priority are part of the patient-state-index, and wherein the infusion is interrupted when patient-state-index is above a threshold value.

In an embodiment, the user selects and/or prioritize the parameters being an at least one additional parameter representing the actual physiological state of the patient and/or being part of the patient-state-index.

In an embodiment, the control unit controls via the actuator at least one infusion parameter, preferably the flow rate and/or the temperature of the infusion fluid, in accordance with the target body temperature, a preset target body temperature, and/or a target body temperature profile.

In an embodiment, the target body temperature profile is a user defined profile, based on at least a preset target body temperature, a change rate of body temperature, and/or target flow rate.

In an embodiment, the control unit controls via the actuator the infusion parameters within at least one upper and/or lower threshold value of the infusion parameters, and/or controls via the actuator the actual body temperature and/or the change rate of the actual body temperature within at least one upper and/or lower threshold value.

In an embodiment, the control unit controls via the actuator the at least one infusion parameter in accordance with at least one upper and/or lower threshold value of the at least one additional parameter, the absorbing capacity, reduction rate and/or patient-state-index or parameters thereof, upper and/or lower threshold values of the change of heart rate, central venous pressure and/or amount of extravascular lungwater.

In an embodiment, no fluid is infused to the patient in the event that at least one additional parameter, at least one infusion parameter, the actual body temperature and/or the change rate of the actual body temperature is/are not within the respective upper and/or lower threshold value(s).

In an embodiment, at least one target value and/or at least one threshold value for the at least one additional parameter, for the at least one infusion parameter, actual body temperature, and/or the change rate of the actual body temperature is/are administered by the user and/or is/are pre-defined data set by the manufacturer.

In an embodiment, the control unit adapts the target body temperature and/or the target temperature profile only when the actual target body temperature and/or the target temperature profile cannot be achieved by a setting of the at least one infusion parameter, which is within the upper and/or lower threshold value(s) for the at least one infusion parameter, and which ensures that the at least one additional parameter and/or the temperature change rate are within the respective upper and/or lower threshold value(s), and/or when the change rate of the actual body temperature is not within its upper and/or lower threshold value.

In an embodiment, in the event of a target body temperature below the actual body temperature, the control unit controls the actuator so as to reduce the flow rate and to reduce the temperature of the infusion fluid thereby keeping the actual body temperature at the target body temperature when the at least one additional parameter, preferably the reduction rate, the absorbing capacity and/or a patient-state-index, and most preferably the change of heart rate, central venous pressure and/or amount of extravascular lungwater, is/are above a first upper threshold value, and/or increase the flow rate and to increase the temperature of the infusion fluid thereby keeping the actual body temperature at the target body temperature when the at least one additional parameter, the reduction rate, the absorbing capacity and/or a patient-state-index, and the change of heart rate, central venous pressure and/or amount of extravascular lungwater, is/are below a first lower threshold value.

In an embodiment, in the event of a target body temperature below the actual body temperature, the control unit controls the actuator so as to reduce the flow rate and to reduce the temperature of the infusion fluid thereby keeping the actual body temperature at a reduced target body temperature when the at least one additional parameter, preferably the reduction rate, the absorbing capacity and/or a patient-state-index), and the change of heart rate, central venous pressure and/or amount of extravascular lungwater, is/are above a second upper threshold value.

In an embodiment, in the event of a target body temperature below the actual body temperature, the control unit stops the supply of infusion to the patient in the event of at least one additional parameter, the reduction rate, the absorbing capacity and/or a patient-state-index, and the change of heart rate, central venous pressure and/or amount of extravascular lungwater, below a third upper threshold value.

In an embodiment, the device triggers an alarm and/or to display an information and/or different status information when the at least one lower and/or upper threshold value is achieved.

In an embodiment, the first lower threshold value is a central venous pressure of 12 cm H2O, the second lower threshold value is a central venous pressure of 20 cm H2O, and the third lower threshold value is a central venous pressure of 35 cm H2O.

In an embodiment, the third upper threshold value is a change of heart rate of 20 beats per minute with a heart beat in the range of 60 to 180 beats per minute.

In an embodiment, the device triggers an alarm and/or to display an information and/or different status information when the amount of infusion left in the supply is below a minimum value, and/or the remaining period of time until the infusion needs to be refilled is below a minimum value.

In an embodiment, the fluid temperature input, the body temperature input, additional input, and/or the flow rate input comprise(s): at least one sensor element, and/or at least one connector for connecting at least one sensor element and/or at least one medical device, wherein the at least one sensor element and/or at least one connector receive, measure, and/or process the actual temperature of the infusion fluid, the actual body temperature of the patient, the at least one additional parameter representing the actual physiological state of the patient and/or the actual flow rate.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a schematic view of one embodiment of the present invention;

FIG. 2 shows a schematic view of another embodiment of the present invention;

FIG. 3 shows a profile of the target body temperature;

FIG. 4 A is a flow chart showing some of the steps carried out by the control unit;

FIG. 4 B is a flow chart showing some of the steps carried out by the control unit;

FIG. 5 is an schematic overview of the additional parameters;

FIG. 6 shows exemplarily the relation between some infusion parameters and some additional parameters, respectively;

FIG. 7 shows a temperature profile of the target body temperature Tb, target which was revised at a given time ta; and

FIG. 8 shows aspects of the flow rate regulation.

### DETAILED DESCRIPTION

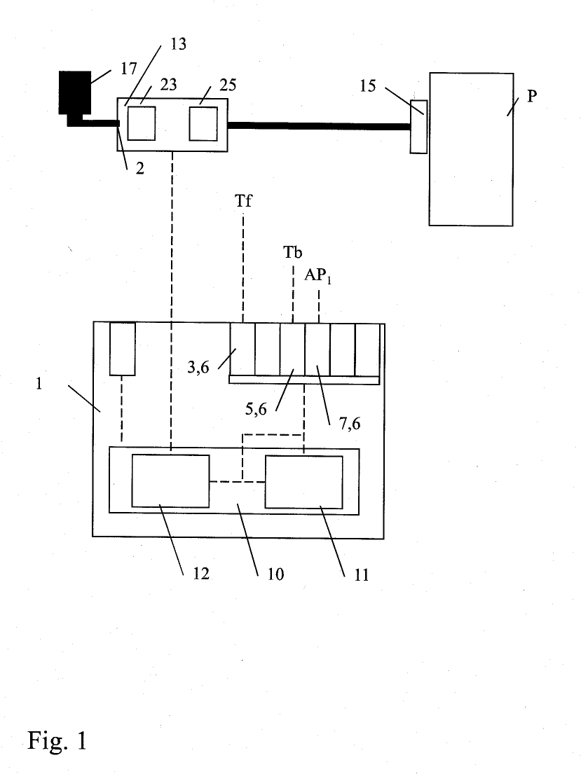

FIG. 1 shows a device 1 for controlling a temperature of a patient by an infusion of fluid. The device 1 comprises a control unit 10 with a memory 11 and a processing unit 12. The processing unit 12 communicates with memory 11 and connectors 6.

In this embodiment, the device comprises seven connectors out of which one is used as a fluid temperature input 3, another one is used as the body temperature input 5 and a third one is used as an additional input 7 which is adapted to receive at least one additional parameter AP representing the actual physiological state of the patient. The connectors 6 could be any kind of standard connector used for connecting any kind of cabling to a device. Alternatively, the connector may be replaced by any suitable wireless data transfer means.

In this embodiment the fluid temperature input 3 receives the actual temperature Tfa of the infusion fluid which may be provided by any suitable sensing means. Body temperature input 5 and additional input 7 receive the actual body temperature Tb and the at least one additional parameter AP representing the actual physiological state of the patient, respectively, by any suitable means and as exemplified before.

The control unit 10 is moreover communicating with an actuator 13 which comprises a temperature actuator 23 and a flow rate actuator 25. The temperature actuator 23 is embodied as a heat exchanger which adjusts the temperature of the infusion fluid. Flow rate actuator 25 is embodied as a pump which adjusts the flow rate of the infusion fluid to be infused.

Device 1 and here actuator 13 has a supply 2 configured as an inlet which is connected via a tube with a reservoir 17 being embodied as an infusion bag. Actuator 13 is moreover connected via a tube to a patient connector 15 which may be an infusion needle as commonly known by the art. Actuator 13 is configured in some embodiments to have or to surround a tube which connects reservoir 17 to connector 15 and the actuators are acting on the infusion fluid from the outside, for instance via a hose pump. The cleaning, sterilization and/or replacement is then advantageously facilitated and the high hygienic requirement are met. In other embodiments two separate tubes are used and the actuators are in direct contact with the infusion fluid.

The control unit 10 controls actuator 13 based on the target body temperature Tb, target the actual temperature of the infusion fluid, the actual body temperature Tb, the actual flow rate FR and one additional parameter AP1. The additional parameter AP1 could be any parameter representing the physiological state of the patient. In this embodiment the one additional parameter is a parameter which represents the absorption capacity of the patient, namely the central-venous pressure. The infusion fluid, thus flows from the infusion bag 17 into actuator 13 in which the temperature and/or the flow rate of the fluid may be adjusted. The infusion fluid may be infused via infusion needle 15.

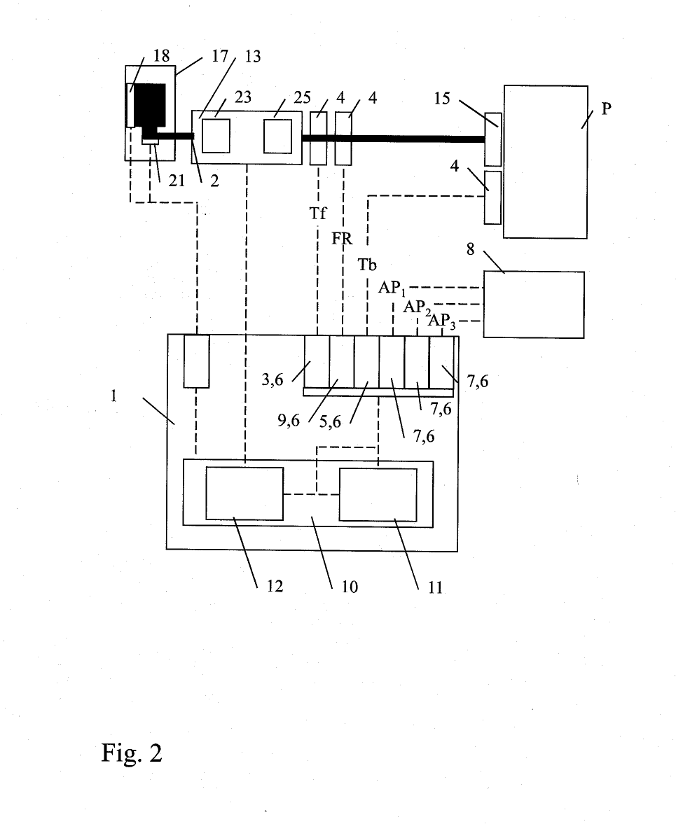

FIG. 2 shows an embodiment with some modifications compared to the embodiment of FIG. 1. Hereafter mainly the differences to the embodiment of FIG. 1 are highlighted. The reference numerals of those parts of FIG. 2 which are identical to those of FIG. 1 are identical.

In FIG. 2 fluid temperature input 3, body temperature input 5 and flow rate input 9 are in direct communication with suitable sensors 4, respectively. Moreover, three additional inputs 7 are connected to a medical device 8 which provides three additional parameter AP1, AP2, AP3 representing the actual physiological state of the patient. In the present embodiment, the three additional parameters AP1, AP2, AP3 are the amount of extra-vascular lung water, the central-venous pressure and the change of heart rate.

The data received by the device 1 are used by control unit 10 to generate a control signal for actuator 13 which adjusts the temperature and/or the flow rate of the infusion fluid to be infused. The embodiment according to FIG. 2 moreover comprises a temperature regulating means 18 such as a heating and/or cooling element which is part of reservoir 17 here embodied as a thermobox.

A conventional infusion bag is positioned in thermobox 17 and connected to a tube which is in communication with inlet 2. Moreover, the thermobox 17 comprises a supply temperature sensor 21 which provides the temperature of the infusion fluid stored in the thermobox 17. The supply temperature sensor 21 and the temperature regulating device 18 are in electronic communication with the control unit 10. In this embodiment, the control unit 10 not only adjusts the temperature and flow rate of the fluid to be infused by actuator 13 but also controls the temperature of the infusion fluid stored in the infusion bag located in thermobox 17.

The temperature of the infusion fluid in the infusion bag is generally regulated to a value which is generally suitable for the infusion and temperature actuator 23 preferably only conducts a final adjustment to the desired temperature of the fluid to be infused. However, it may also be possible to configure the temperature actuator 23 and the temperature regulating device 18 so that the workload is equally shared among them.

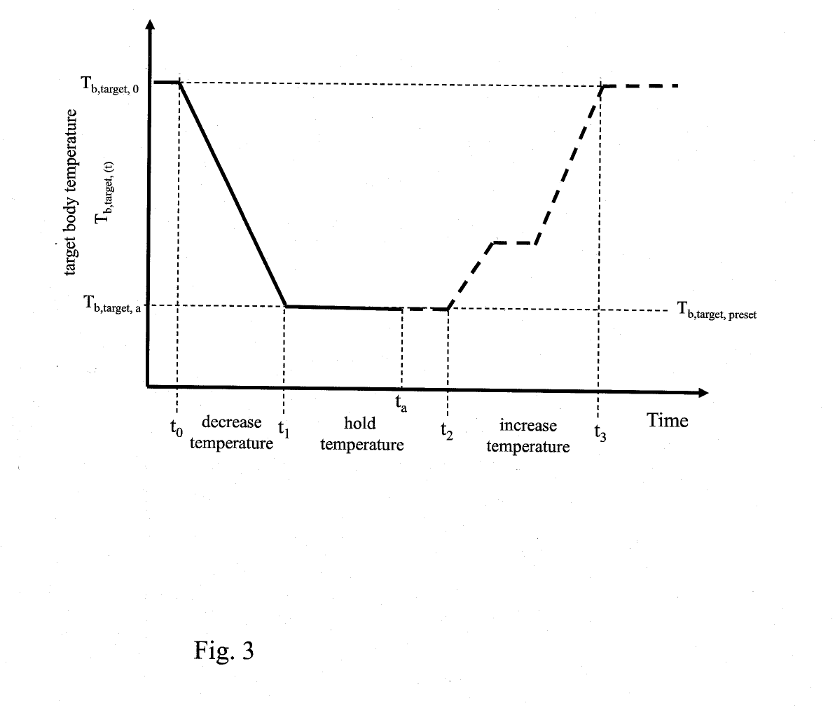

FIG. 3 shows a temperature profile of the target body temperature Tb, target (t). The temperature control generally starts with an initial target body temperature Tb, target, 0 which is equal to the actual body temperature Tb of the patient. The target body temperature then decreases with a preset change rate of body temperature until a lower preset target body temperature Tb, target, preset is reached at a time t1. The preset target body temperature Tb, target, preset will be constant until a given time t2. Afterwards, the target body temperature Tb, target is herein increased in two steps. The treatment is finished at a time t3 in which the target body temperature Tb, target is equal to the body temperature of the patient without temperature control.

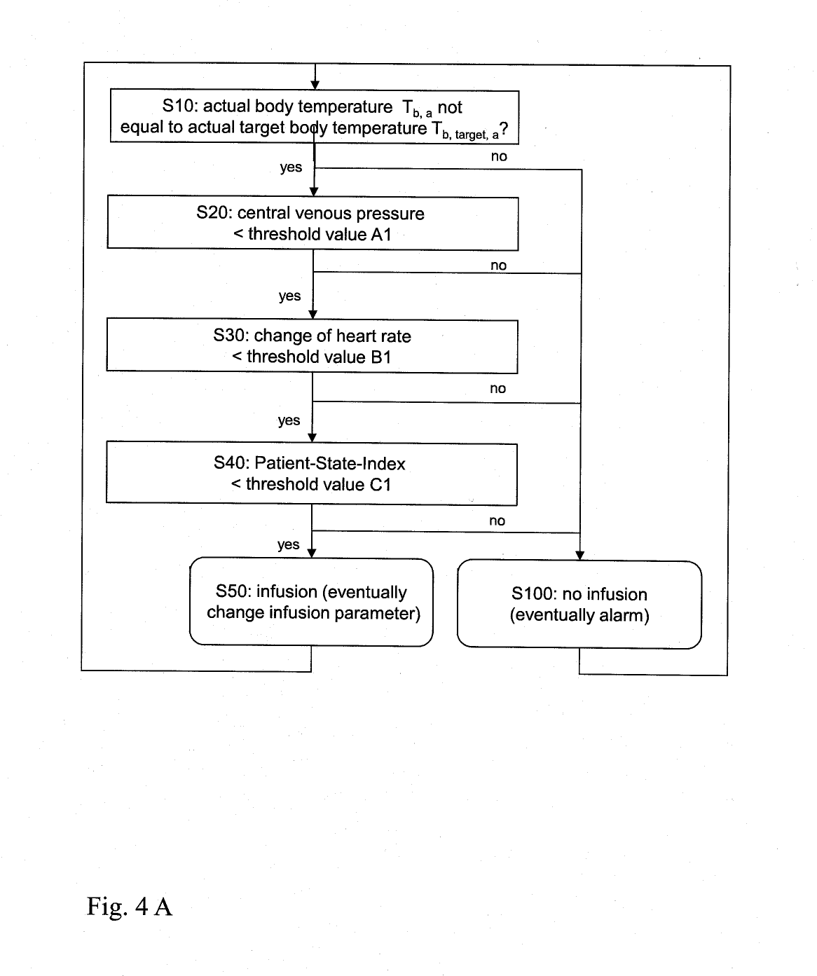

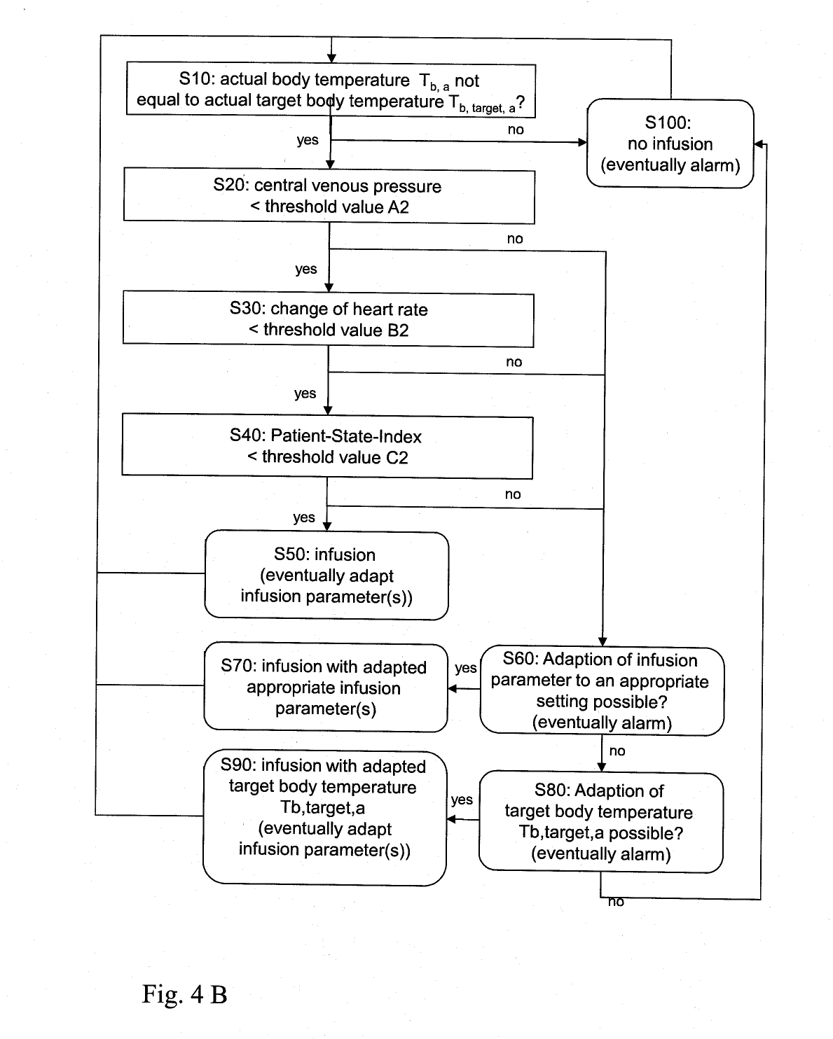

FIG. 4a depicts some of the steps carried out by control unit 10 in order to control the temperature of a patient by an infusion of a fluid. In step S10, the actual body temperature Tb, a in a given point to as, for instance, shown in FIG. 3 is compared to an actual target body temperature Tb, target, a. If the actual body temperature Tb, a is not equal to the actual target body temperature Tb, target, a then the central venous pressure is compared to a threshold value A of the central-venous pressure in a step S20. If the central venous pressure is lower than the respective threshold value A then the change of heart rate is compared to a threshold value B of the change of heart rate in a step S30. If the change of heart rate is lower than a threshold value B then a patient-state-index is compared to a threshold value C of the patient state index in step S40. If the patient-state-index is lower that the respective threshold value C then the infusion will be carried out in step S50. Eventually, one or several infusion parameters, here the temperature of the fluid to be infused and/or the flow rate FR are adjusted in step S50 and the control unit 10 restarts the routine with S10.

If in the steps S20, S30, S40 any of the values for the central venous pressure, change of heart rate, and/or patient-state-index is above the respective threshold value A1, B1 and/or C1 no infusion fluid will be infused in step S100 and, eventually, the device will trigger an alarm.

FIG. 4b also depicts some of the steps carried out by control unit 10 in order to control the temperature of a patient by an infusion of a fluid. The steps S10, S20, S30, S40, S50 are identical to those of the embodiment in accordance to FIG. 4a.

If in the steps S20, S30, S40 any of the values for the central venous pressure, change of heart rate, and/or patient-state-index is above the respective threshold value A2, B2 and/or C2 the control unit 10 does not immediately stop the infusion fluid but evaluates in step S60 whether the infusion parameter(s) may be adapted to an appropriate setting, which further allows the infusion of fluid within the threshold parameter A2, B2, C2 and eventually within the threshold value(s) for the infusion parameter(s). The flow rate FR may, for instance, be decreased to reduce the central venous pressure and the temperature of the fluid may be further decreased at the same time in order to obtain the same body temperature regulating effect in the event of cooling a patient. If the infusion with an adapted appropriate infusion parameter setting is possible, then the infusion continues with an adapted appropriate infusion parameter setting in step S70 and the control unit 10 restarts the routine with S10.

If the infusion with an adapted appropriate infusion parameter setting is not possible, then the control unit 10 evaluates in step S80 whether the infusion may be possible with an adapted target body temperature Tb, target, a, eventually together with an adapted appropriate infusion parameter setting. For instance, in the event of cooling a patient, the required flow rate FR may be further reduced if the adapted target body temperature Tb, target, a is closer to the normal body temperature of the uncooled patient which would additionally decrease the central venous pressure. If the infusion is possible with an adapted target body temperature Tb, target, a, eventually together with an adapted appropriate infusion parameter setting then the infusion continues with an adapted target body temperature Tb, target, a, eventually together with an adapted appropriate infusion parameter setting in step S90 and the control unit 10 restarts the routine with S10. If an infusion with an adapted target body temperature Tb, target, a, eventually together with an adapted appropriate infusion parameter setting, is not possible then no fluid is infused in step S100). The control unit moreover eventually triggers an alarm during step S60, S80, and/or S100.

The embodiments of FIG. 4a and FIG. 4b could also be combined. For instance with threshold values A2, B2, C2 which are lower than threshold values A1, B1, C1. In such an embodiment, the control unit 10 may first evaluate an appropriate infusion parameter setting and/or an adapted target body temperature Tb, target, a, if any one of the threshold values A2, B2, C2 is reached and may only stop the infusion, if no appropriate infusion parameter setting and no adapted target body temperature Tb, target, a is possible or the threshold values A1, B1, C1 are reached, provided that the actual body temperature Tb, a is not equal to the actual target body temperature Tb, target, a.

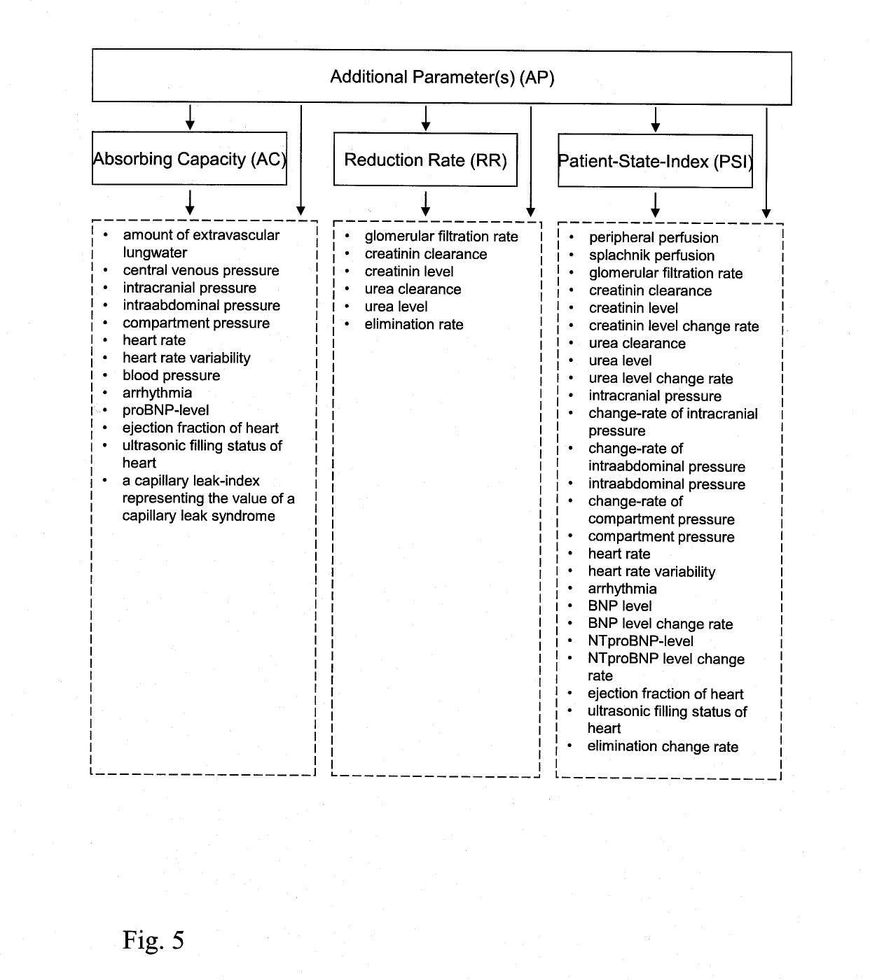

FIG. 5 shows exemplarily some of the additional parameters which represents the actual physiological state of the patient. The additional parameters may be subdivided into three groups. The first group concerning the absorbing capacity AC represents the patient's ability to absorb infusion fluid. The second group is the reduction rate RR which represents parameters indicative for the patient's ability to reduce fluid contained in the body. A third group of parameters are indicative for the general condition of the patient which are in the present embodiment associated to a patient-state-index.

The patient-state-index is here an index which considers several parameters for the calculation of the patient-state-index value. The patient-state-index value has here the same priority as the reduction rate RR and/or the absorbing capacity AC, for instance exemplified by the central-venous pressure and/or the change of heart rate. It is also possible that the control unit 10 only considers one or several additional parameters AP without calculating a patient-state-index PSI. The controller might, for instance, evaluate in a step S40 of FIG. 4a or 4b not the patient-state-index but the amount of extra-vascular lung water. Also, the amount of extra-vascular lung water could be evaluated in an additional step S45.

The vertical arrows in FIG. 5 symbolize that each parameter listed within the respective rectangles with a broken line could be an additional parameter AP. Moreover each parameter listed in the left rectangle with a broken line could represent an absorbing capacity AC, each parameter listed in the center rectangle with a broken line could represent an reduction rate RR, and each parameter listed in the right rectangle with a broken line could represent a parameter for the calculation of the patient-state-index.

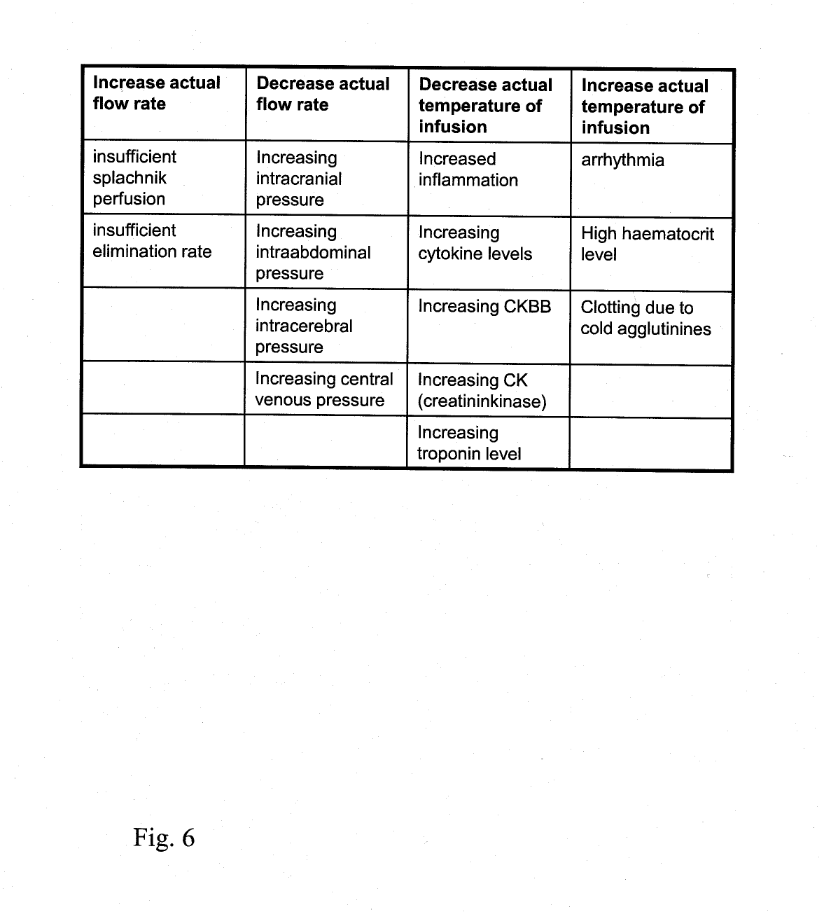

FIG. 6 shows some of the additional parameters AP and their impact on the infusion parameters flow rate FR and/or temperature of infusion fluid.

The splanchnic perfusion may be an indicator for the actual volume status and/or peripheral resistance. Knowing the volume status and/or the peripheral resistance is vital for controlling the body temperature by infusion. If the splanchnic perfusion is insufficient the flow rate FR should preferably be increased.

The elimination rate indicates how much physiological waste can be eliminated in a specific period of time. It also partly indicates the metabolism of the body. An insufficient elimination rate would require the decrease the actual flow rate.

The intra-cranial pressure is dependent on several factors such as the heart capacity, the volume status, the blood pressure, edema etc. The intra-cranial pressure is influenced by the infusion of fluid and should not be increased above a certain value. Thus, the actual flow rate FR should be decreased if the intra-cranial pressure is increased or increasing.

Similar to intra-cranial pressure, the intra-cerebral pressure depends on several factors such as heart capacity, volume status, blood pressure, edema, etc. Thus, it is important not to increase the intra-cerebral pressure above a certain limit. Consequently, the actual flow rate FR of the fluid should be decreased when the intra-cerebral pressure is increased or increasing.

The intra-abdominal pressure may be an indication for the volume status of a patient especially in cases of ascites. It is therefore important not to increase the intra-abdominal pressure to much by an infusion in order to avoid complications like the compartment syndrome. Therefore, the actual flow rate FR of the infused fluid should be decreased in the case of an increased or increasing intra-abdominal pressure.

The central-venous pressure is an indicator for the volume status of the body and the volume capacity of the heart. A increased or increasing central-venous pressure indicates a limited capacity of the body for further absorption of infusion fluid. Thus, the actual flow rate FR of the infusion fluid to be infused into the patient should be reduced when a central-venous pressure is increased or increasing.

Troponin is an indicator for heart cell death. In order to avoid heart cell death, the actual flow rate should be decreased in the case of an increasing troponin level. Alternatively or additionally, the actual temperature of the infusion may be decreased in the case of an increased or increasing troponin level.

Inflammatory processes may be influenced by the temperature of the infused fluid. By lowering the temperature, it is possible to significantly decelerate the inflammation and prevent inflammatory primary and/or secondary damages. Thus, the actual temperature of the infused fluid may be decreased in the case of an increased or increasing inflammation and vice-versa.

Cytokine levels are inter alia an indication for inflammation, cell death, infection, sepsis, etc. By lowering the temperature it is possible to significantly slower the metabolism and the results of cytokines, and cytokine production. Thus, the actual temperature of the infusion fluid may be decreased in the case of an increased or increasing cytokine levels.

Arhythmia may be an indicator for disturbed signal transmission due to a low temperature of the body. Since it is important not to disturb the signal transmission the actual temperature of the infusion should be increased when arrhythmia occurs.

Lowering the temperature the body leads to a higher viscosity which may lead to clotting. Since it is important to keep the blood fluidly enough the actual temperature of the infused fluid may be increased in the case of an increased or increasing haematocrite level.

Some patients may have cold agglutinines which would lead to intra-vasal clotting. In order to avoid intra-vasal clotting the actual temperature of the infusion may be increased in the case of clotting due to cold agglutinines.

The functional relationship between the infusion parameter(s) and aforementioned parameters such as, for instance, the central venous pressure are described herein only in one direction, for instance, that the flow rate should be decreased in the case of an increasing or increased central venous pressure. It is readily understood, that the same functional relationship applies inversely, for instance that the flow rate could be increased in the case of a low or non critical central venous pressure.

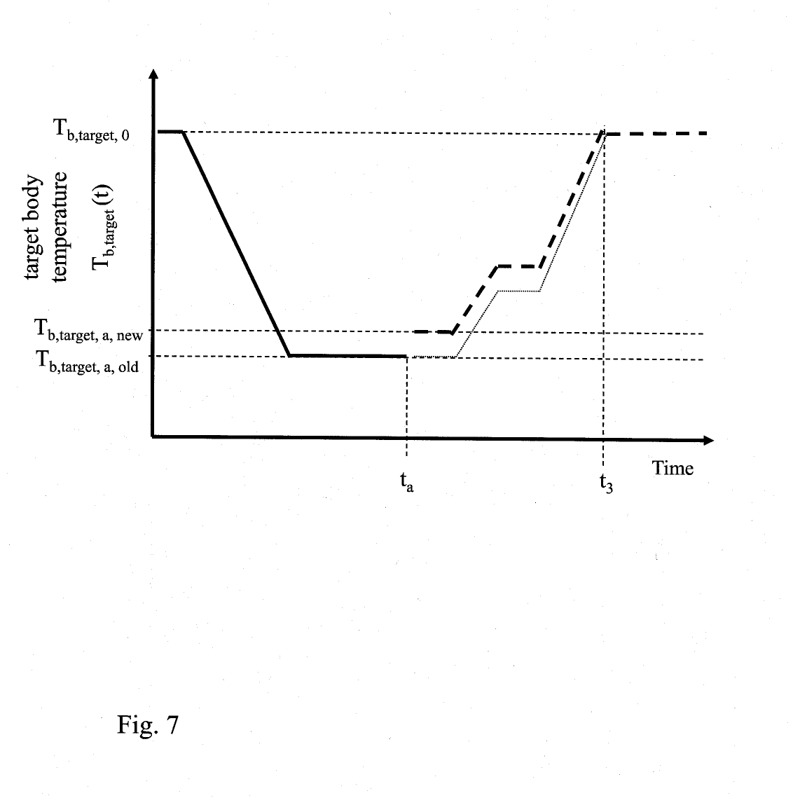

FIG. 7 shows a target body temperature Tb, target (t) as a function of time. The solid bold line representing the target body temperature Tb, target (t) until a point of time ta and a dotted line representing the target body temperature Tb, target (t) until a point of time t3 correspond to the target body temperature profile of FIG. 3. The control unit 10 could not change the infusion parameter(s) at the point of time ta to an appropriate setting so that the infusion parameter(s) and/or the additional parameter(s) AP are within the respective threshold value (s). For instance, the control unit 10 could not find a setting in step S60 of FIG. 4b which ensures that a central venous pressure is below a threshold value A2 as evaluated in step S20 of FIG. 4b and/or a change of heart rate being below a threshold value B2 as evaluated in step S30 of FIG. 4b and/or a patient-state-index PSI which is below a threshold value C2 as evaluated in step S40 of FIG. 4b. In other words, instead of stopping the infusion as done in step S100 of FIG. 4a, the control unit 10 calculates a new target body temperature Tb, target, a, new and thus a new temperature profile which is depicted as a bold broken line in FIG. 7. The target body temperature Tb, target, a, new as well as the new target body temperature profile are calculated by the control unit 10 so that the threshold values of the infusion parameter(s) and/or of the additional parameter(s) AP will presumably not be reached. Additionally, the device will inform the user about the new target body temperature profile.

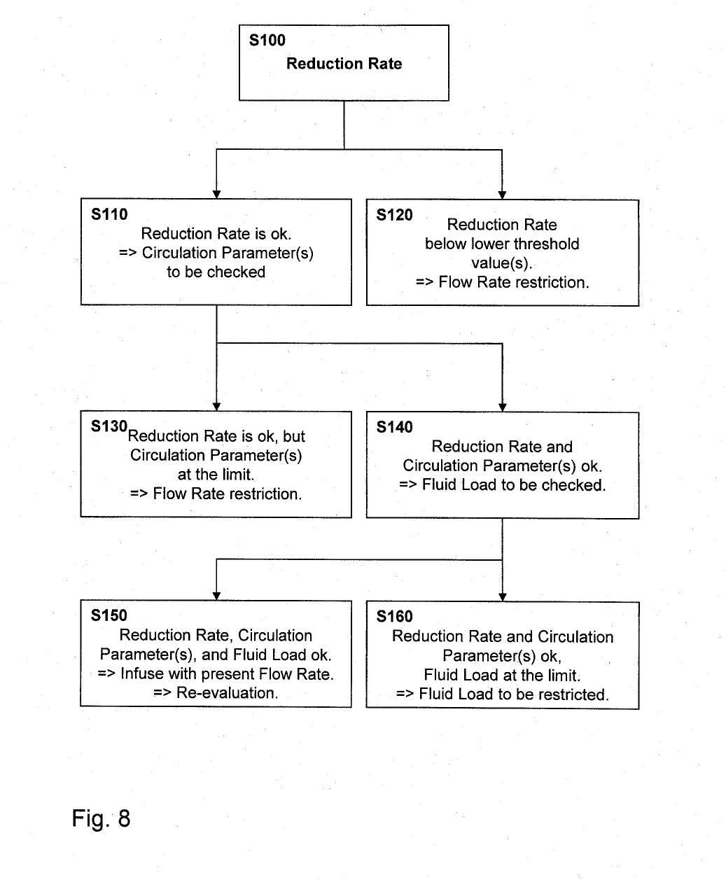

FIG. 8 shows an exemplary embodiment of the control method. At step S100 the Reduction Rate is evaluated. If the reduction rate is ok and does not require any restriction of the flow rate FR since the reduction rate is below a lower threshold value, then the circulation parameter(s) is/are to be checked (S110), otherwise the flow rate FR will be restricted (S120). If the reduction rate and the circulation parameter(s) are ok and do not require any restriction of the flow rate FR, then the fluid load is to be checked (S140), otherwise the flow rate FR will be restricted (S130). If the reduction rate, the circulation parameter(s) and the fluid load are ok and do not require any restriction of the flow rate FR, then the fluid is infused with the actual flow rate (S150), otherwise the flow rate FR will be restricted (S160).

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality.

The invention also covers all further features shown in the figures individually although they may not have been described in the afore description. The present invention covers further embodiments with any combination of features from different embodiments described above. Reference signs in the claims are provided merely as a clarifying example and shall not be construed as limiting the scope of the claims in any way. The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “essentially radial” shall also cover exactly radial).

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A device for controlling or assisting to control a temperature of a patient by infusion of fluid, the comprising:
at least one supply of infusion fluid;
at least one body temperature input adapted to receive an actual body temperature of the patient;
at least one additional input adapted to receive at least one additional parameter representing an actual physiological state of the patient;
at least one control unit communicating with the body temperature input and the additional input; and
at least one actuator which controls at least the actual flow rate and/or actual temperature of the infusion fluid in accordance with at least one control signal of the control unit,
wherein the control unit controls the temperature of a patient with the actuator based on at least a target body temperature, the actual temperature and/or the defined temperature of the infusion fluid, the actual body temperature, the actual flow rate, and at least one additional parameter representing the actual physiological state of the patient including the change of heart rate, and
wherein, in an event of a target body temperature being below the actual body temperature, the control unit is adapted to control the actuator so as to reduce the flow rate and to reduce a temperature of the infusion fluid thereby keeping the actual body temperature at the target body temperature when at least one additional parameter is above a first upper threshold value, or to increase the flow rate and to increase the temperature of the infusion fluid thereby keeping the actual body temperature at the target body temperature when at least one additional parameter is below a first lower threshold value.

2. The device according to claim 1, wherein at least one further additional parameter is the amount of extravascular lungwater and/or central venous pressure.

3. The device according to claim 1, wherein a further additional parameter is a reduction rate representing the patient's capacity to reduce fluid in the body.

4. The device according to claim 3, wherein the reduction rate includes one or several of the parameters indicating the elimination of physiological waste, such as glomerular filtration rate, creatinin clearance, creatinin level, urea clearance, urea level, elimination of physiological waste, and/or elimination rate.

5. The device according to claim 1, wherein an additional parameter is a first or second time derivative of a physiological parameter.

6. The device according to claim 1, wherein the device comprises at least one fluid temperature input communicating with the control unit and adapted to receive the actual temperature of the infusion fluid, and/or a defined value for the temperature of the infusion fluid.

7. The device according to claim 1, wherein the device comprises at least one flow rate input communicating with the control unit and adapted to receive the actual flow rate of the infusion fluid, and/or wherein the device derives the actual flow rate of the infusion fluid based on the settings of actuator.

8. The device according to claim 1, wherein the control unit comprises:
a memory adapted to store data; and/or
a data processing unit adapted to read the data of the memory and/or of the inputs and process the data to a control signal for the actuator.

9. The device according to claim 1, wherein the target body temperature is a preset target body temperature and/or a target body temperature profile.

10. The device according to claim 9, wherein the target body temperature profile is a user defined profile, based on at least a preset target body temperature, a change rate of body temperature, and/or target flow rate.

11. The device according to claim 1, wherein the control unit controls, via the actuator, the infusion parameters flow rate and/or actual temperature of the infusion fluid within at least one upper and/or lower threshold value of the infusion parameters, and/or controls, via the actuator, the actual body temperature and/or the change rate of the actual body temperature within at least one upper and/or lower threshold value.

12. The device according to claim 1, wherein the control unit adapts the target body temperature and/or the target body temperature profile when the actual target body temperature and/or the target body temperature profile cannot be achieved by a setting of the infusion parameters, which are within the upper and/or lower threshold value(s) for the infusion parameters, and which ensures that the at least one additional parameter and/or the temperature change rate are within the respective upper and/or lower threshold value(s), and/or when the change rate of the actual body temperature is not within its upper and/or lower threshold value.

13. The device according to claim 1, wherein at least one target value and/or at least one threshold value for the at least one additional parameter, for the infusion parameters, for the actual body temperature, and/or for the change rate of the actual body temperature is/are administered by the user and/or is/are pre-defined data set by the manufacturer.

14. The device according to claim 1, wherein, in the event of a target body temperature below the actual body temperature, the control unit is adapted to control the actuator so as to reduce the flow rate and to reduce the temperature of the infusion fluid thereby keeping the actual body temperature at a reduced target body temperature when at least one additional parameter is/are above a second upper threshold value.

15. The device according to claim 1, wherein, in the event of a target body temperature below the actual body temperature, the control unit is adapted to stop the supply of infusion to the patient in the event of at least one additional parameter above a third upper threshold value.

16. The device according to claim 15, wherein the third upper threshold value is a change of heart rate of 20 beats per minute with a heart beat in the range of 60 to 180 beats per minute.
